(19) United States
(12) Patent Application Publication
(10) Pub. No.: US 2014/0243872 A1
(43) Pub. Date: Aug. 28, 2014

(54) REUSABLE SINUS DILATION INSTRUMENT AND METHOD OF USE

(71) Applicant: Scott Cordray, Tulsa, OK (US)

(72) Inventor: Scott Cordray, Tulsa, OK (US)

(21) Appl. No.: 14/271,913

(22) Filed: May 7, 2014

Related U.S. Application Data
(60) Provisional application No. 61/915,791, filed on Dec. 13, 2013.

(51) Int. Cl. A61M 29/02

(52) U.S. Cl. CPC A61M 29/02; A61M 2029/025
USPC 606/192

(57) ABSTRACT

The invention is directed to a system and method for a less invasive surgical tool and operation to correct sinus disease called balloon sinuplasty. The reusable sinus dilation instrument has a guide seeker constructed from a sterilizable material with a bended terminal end offset from an attachment terminal end and a central grip intermediate of the terminal ends. The instrument also includes a removable handle capable of being attached to the attachment terminal end at a predetermined orientation relative to the bended terminal end of the guide seeker depending upon if the instrument is a frontal sinus guide seeker, a maxillary sinus guide seeker or a sphenoid sinus guide seeker. The bended terminal end of the guide seeker can be selectively angled so that the guide seeker is a frontal sinus guide seeker, a maxillary sinus guide seeker or a sphenoid sinus guide seeker.

### CROSS REFERENCE TO RELATED APPLICATIONS

This application claims priority to and the benefit of U.S. Provisional Patent Application No. 61/915,791, filed Dec. 13, 2013, which is incorporated herein by reference in its entirety.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### REFERENCE TO A SEQUENCE LISTING, A TABLE, OR A COMPUTER PROGRAM LISTING APPENDIX

Not Applicable.

### BACKGROUND OF THE INVENTION

1. Field of the Invention

This invention relates generally to a reusable sinus dilation instrument and method of use, and more particularly to a reusable sinus dilation instrument having a guide seeker constructed from a sterilizable material with a bended terminal end offset from an attachment terminal end and a central grip intermediate of the terminal ends, and having a removable handle capable of being attached to the attachment terminal end at a predetermined orientation relative to the bended terminal end of the guide seeker.

2. Description of the Related Art

Sinus disease often occurs because of blockage. The blockage traps mucous in a warm, moist environment and can produce infection. Blockage also allows pressure to build which can cause pain and pressure over the sinus cavities. Balloon sinuplasty is a newer less invasive operation to correct sinus disease. During the procedure, no mucosal tissue is removed, rather the ostia or opening on the sinus is enlarged by inserting a balloon that is inflated. The opening then heals in the open position. Balloon sinuplasty overcomes the infection, pain, and pressure by keeping the sinus ostia open.

Current medical-grade instruments used in balloon sinuplasty have a high cost because most of the parts are intended for one time only use. There are less expensive instruments, but those instruments have problems with bulk and awkward angles, which make these instruments very difficult to use and can cause more tissue trauma, which may lead to more scarring and revisions.

It is therefore desirable to provide an improved reusable sinus dilation instrument and method of use.

It is further desirable to provide a reusable sinus dilation instrument that can be safely sterilized, has better angles for finding the sinus, and has less profile so as to cause less tissue trauma.

It is still further desirable to provide a reusable sinus dilation instrument having a guide seeker and a disposable balloon capable of being slipped over the guide seeker.

It is yet further desirable to provide a reusable sinus dilation instrument having a guide seeker with a bended terminal end offset from an attachment terminal end and a central grip intermediate of the terminal ends.

It is still yet further desirable to provide a reusable sinus dilation instrument having a removable handle capable of being attached to an attachment terminal end at a predetermined orientation relative to a bended terminal end of a guide seeker of the instrument.

It is yet further desirable to provide a reusable sinus dilation instrument where the angle of the terminal bend varies depending on the sinus being treated, e.g., about 45 degrees for frontal, about 95 degrees for maxillary, and about 10 degrees for sphenoid sinus.

It is still yet further desirable to provide a reusable sinus dilation instrument having a fiber optic cable in the center of the guide seeker.

It is still yet further desirable to provide a reusable sinus dilation instrument made of sterilizable material, e.g. stainless steel.

It is still yet further desirable to provide a reusable sinus dilation instrument having a removable handle capable of being attached to the instrument using a luer taper fitting, a threaded attachment or other attachment means.

Other advantages and features will be apparent from the following description and from the claims.

### SUMMARY OF THE INVENTION

In general, the invention relates to a reusable sinus dilation instrument and method of use for less invasive balloon sinuplasty for correction of sinus disease. The instrument may be sterilized and reused, has better angles for finding the sinus and causes less tissue trauma. The instrument includes a guide seeker having a bended terminal end offset from an attachment terminal end and a central grip intermediate of the terminal ends. The instrument also includes a removable handle attachable to the attachment terminal end at a predetermined orientation relative to the bended terminal end of the guide seeker. The bended terminal end may be approximately 2.5 cm length, and has an angle that varies depending on sinus being treated—approximately 45 degrees for frontal, approximately 95 degrees for maxillary, and approximately 10 degrees for sphenoid sinus. In addition, a fiber optic cable may be placed in the center of the guide seeker. The guide seeker may be made of sterilizable material, e.g. stainless steel. The removable handle may be attached to the instrument using a luer taper fitting, a threaded attachment or other attachment means.

### BRIEF DESCRIPTION OF THE DRAWINGS

FIG. 1 is a top plan view of an example of a reusable sinus dilation instrument having a frontal sinus guide seeker and a removable handle oriented generally coplanar with and parallel to a bended terminal end of the guide seeker in accordance with an illustrative embodiment of the invention disclosed herein;

FIG. 2 is a side view of the instrument shown in FIG. 1;

FIG. 3 is an exploded view of the instrument shown in FIG. 2;

FIG. 4 is an exploded view of area 4 as shown in FIG. 2;

FIG. 5 is a side view of an example of a reusable sinus dilation instrument having a disposable balloon capable of being slipped over a bended terminal end of a guide seeker in accordance with an illustrative embodiment of the invention disclosed herein;

FIG. 6 is a side view of another example of a reusable sinus dilation instrument having a disposable balloon capable of being slipped over an attachment terminal end of a guide seeker in accordance with an illustrative embodiment of the invention disclosed herein;

FIG. 7 is a side view of the reusable sinus dilation instrument shown in FIGS. 5 and 6 with the disposable balloon seated over the guide seeker;

FIG. 8 is a side fragmentary view of an example of a maxillary sinus guide seeker in accordance with an illustrative embodiment of the invention disclosed herein;

FIG. 9 is a side fragmentary view of the maxillary sinus guide seeker shown in FIG. 8 with a disposable balloon seated on a bended terminal end;

FIG. 10 is a side fragmentary view of an example of a sphenoid sinus guide seeker in accordance with an illustrative embodiment of the invention disclosed herein;

FIG. 11 is a side fragmentary view of the sphenoid sinus guide seeker shown in FIG. 10 with a disposable balloon seated on a bended terminal end;

FIG. 12 is a side view of another example of a reusable sinus dilation instrument having a removable handle attachable a frontal sinus guide seeker at a predetermined orientation in accordance with an illustrative embodiment of the invention disclosed herein;

FIG. 13 is a side view of the reusable sinus dilation instrument shown in FIG. 12 with the removable handle attached to the guide seeker and oriented generally coplanar with and in an opposing parallel direction to a bended terminal end of the frontal sinus guide seeker; and

FIG. 14 is a perspective view of another example of a reusable sinus dilation instrument having a maxillary sinus guide seeker and a removable handle disposed generally perpendicular to a bended terminal end of the guide seeker in accordance with an illustrative embodiment of the invention disclosed herein.

### DETAILED DESCRIPTION OF THE INVENTION

The instruments and methods discussed herein are merely illustrative of specific manners in which to make and use this invention and are not to be interpreted as limiting in scope.

While the invention has been described with a certain degree of particularity, it is to be noted that many modifications may be made in the construction and the arrangement of its components without departing from the scope of the invention. It is understood that the invention is not limited to the embodiments set forth herein for purposes of exemplification.

The description of the invention is intended to be read in connection with the accompanying drawings, which are to be considered part of the entire written description of this invention. In the description, relative terms such as “front,” “rear,” “lower,” “upper,” “horizontal,” “vertical,” “above,” “below,” “up,” “down,” “top” and “bottom” as well as derivatives thereof (e.g., “horizontally,” “downwardly,” “upwardly” etc.) should be construed to refer to the orientation as then described or as shown in the drawings under discussion. These relative terms are for convenience of description and do not require that the machine be constructed or the method to be operated in a particular orientation. Terms, such as “connected,” “connecting,” “attached,” “attaching,” “join” and “joining” are used interchangeably and refer to one structure or surface being secure to another structure or surface or integrally fabricated in one piece.

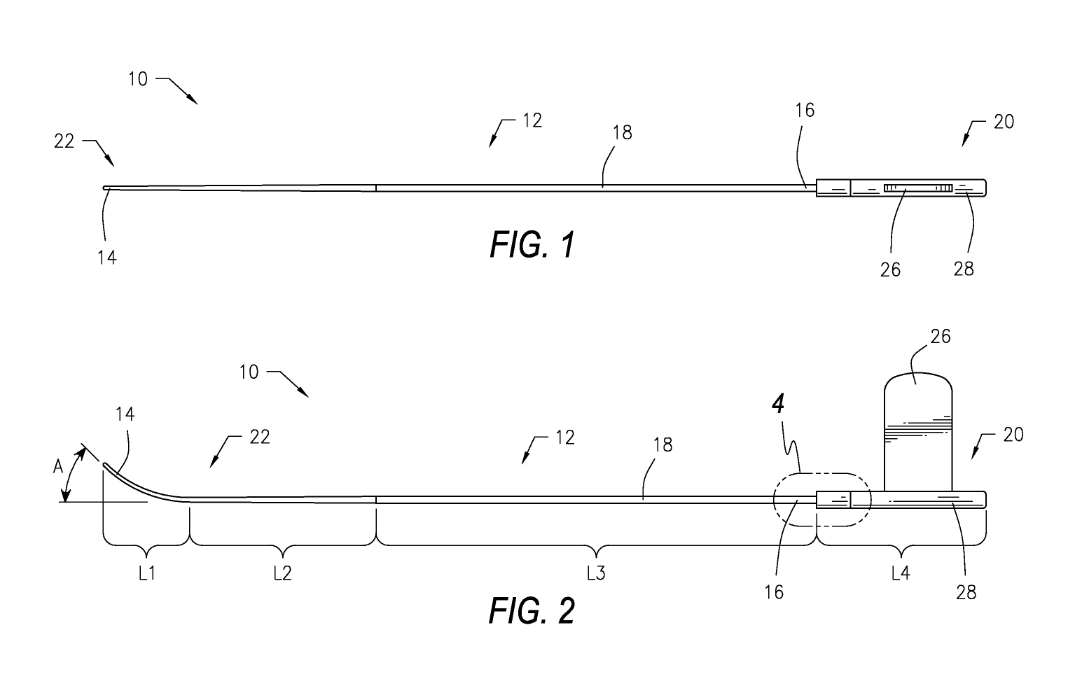

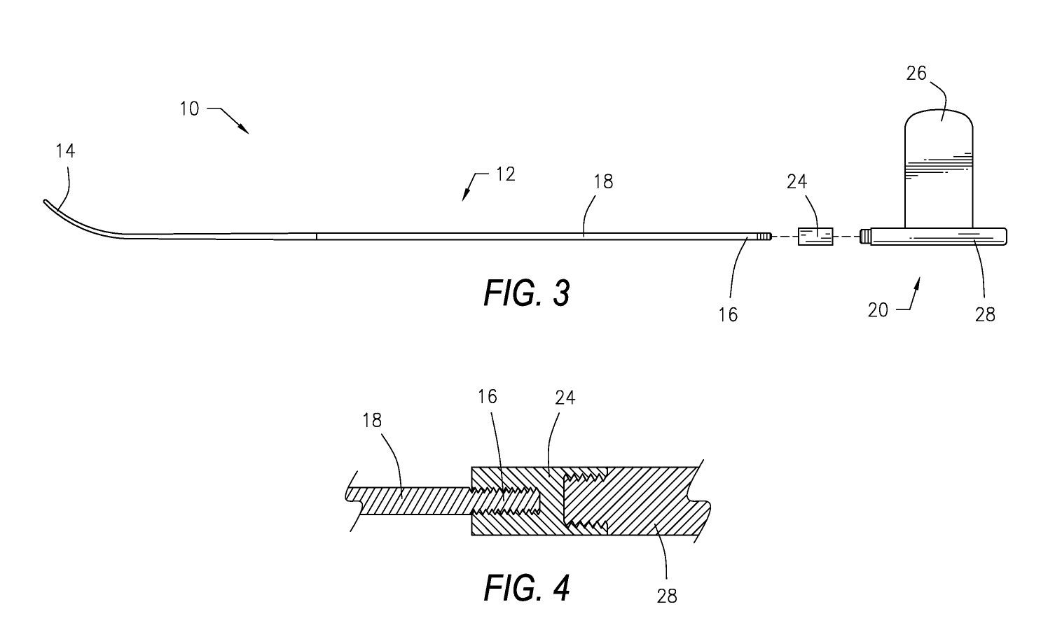

Referring to the figures of the drawings, wherein like numerals of reference designate like elements throughout the several views, and initially to FIGS. 1 through 7, a reusable sinus dilation instrument 10 has a guide seeker 12 constructed from a sterilizable material, such as stainless steel. The guide seeker 12 includes a bended terminal end 14 offset from an attachment terminal end 16 and a central grip 18 intermediate of the terminal ends 14/16. The instrument 10 also includes a removable handle 20 capable of being attached to the attachment terminal end 16 at a predetermined orientation relative to the bended terminal end 14 of the guide seeker 12.

As exemplified in FIGS. 1 through 3 and 5 through 7, the guide seeker 12 is a frontal sinus guide seeker 12 with the removable handle 20 oriented generally coplanar with and parallel to the bended terminal end 14 of the guide seeker 12. The bended terminal end 14 of the frontal sinus guide seeker 12 may have an angle A of about 45 degrees with a long gentle curve of approximately 2.5 cm in length L1. A tip 22 of the curve of the bended terminal end 14 may be approximately 1 mm diameter and progressively enlarge to about 2 mm in diameter over a length L2 of about 8 cm. The length L3 of the central grip 18 of the guide seeker 12 continues the diameter of approximately 2 mm to the attachment terminal end 16 of the guide seeker 12.

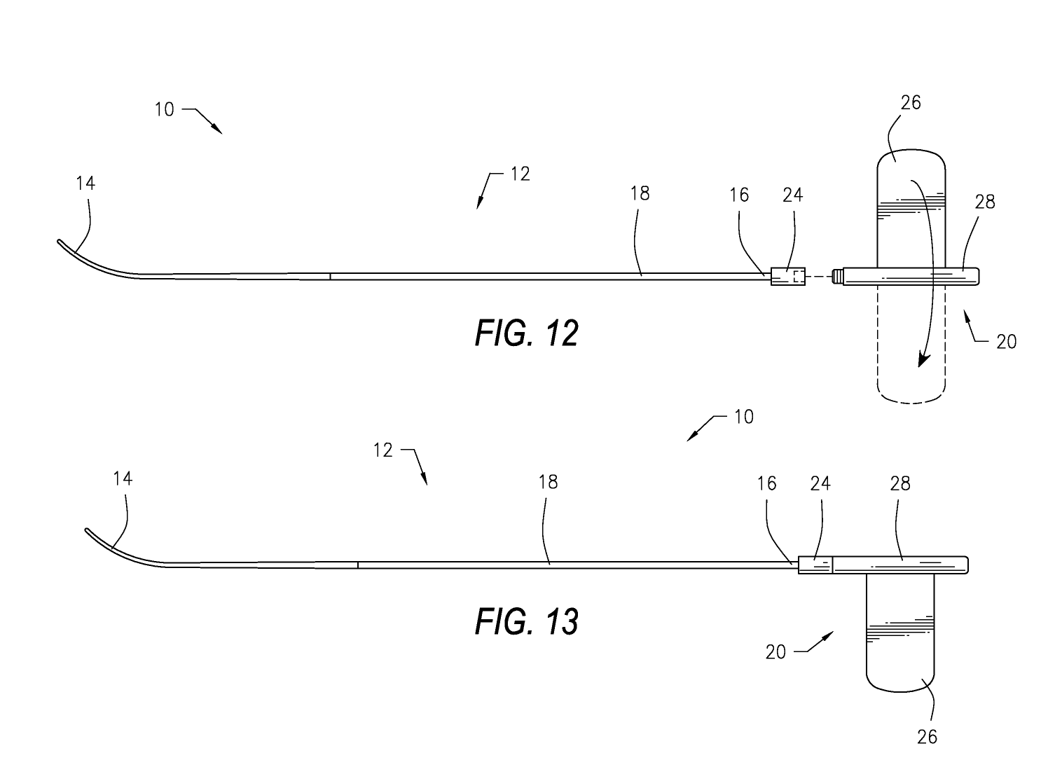

The removable handle 20 of the instrument 10 is capable of being attached to the attachment terminal end 16 at a predetermined orientation relative to the bended terminal end 14 of the guide seeker 12. The handle 20 of the instrument 10 may increase in diameter to about 5 mm and be about 2 cm in length L4. Similarly to the guide seeker 12, the handle 20 may be constructed of a sterilizable material, such as stainless steel. As depicted in the figures, namely FIG. 4, the handle 20 is coupled to the attachment terminal end 16 of the guide seeker 12 using a threaded attachment mechanism 24; however, the invention is not so limited as the handle 20 can be attached to the instrument 10 using a luer taper fitting or other suitable attachment means. The handle 20 may include a wing 26 attached to and protrude away from a body 28 of the handle 20. The wing 26 may be about 2 mm thick, about 3.5 cm long and about 2 cm wide, resulting in the instrument 10 having a length of about 23 cm from the tip 22 of the bended terminal end 14 of the guide seeker 12. As illustrated in FIGS. 1 through 3 and 5 through 7, the handle 20 is oriented generally coplanar with and parallel to the bended terminal end 14 of the front sinus guide seeker 12. Alternatively as illustrated in FIGS. 12 and 13, the handle 20 may be selectively attached to the attachment terminal end 16 of the guide seeker 12 such that the wing 26 of the handle 20 projects in a direction opposite of the curve of the bended terminal end 14 of the guide seeker 12.

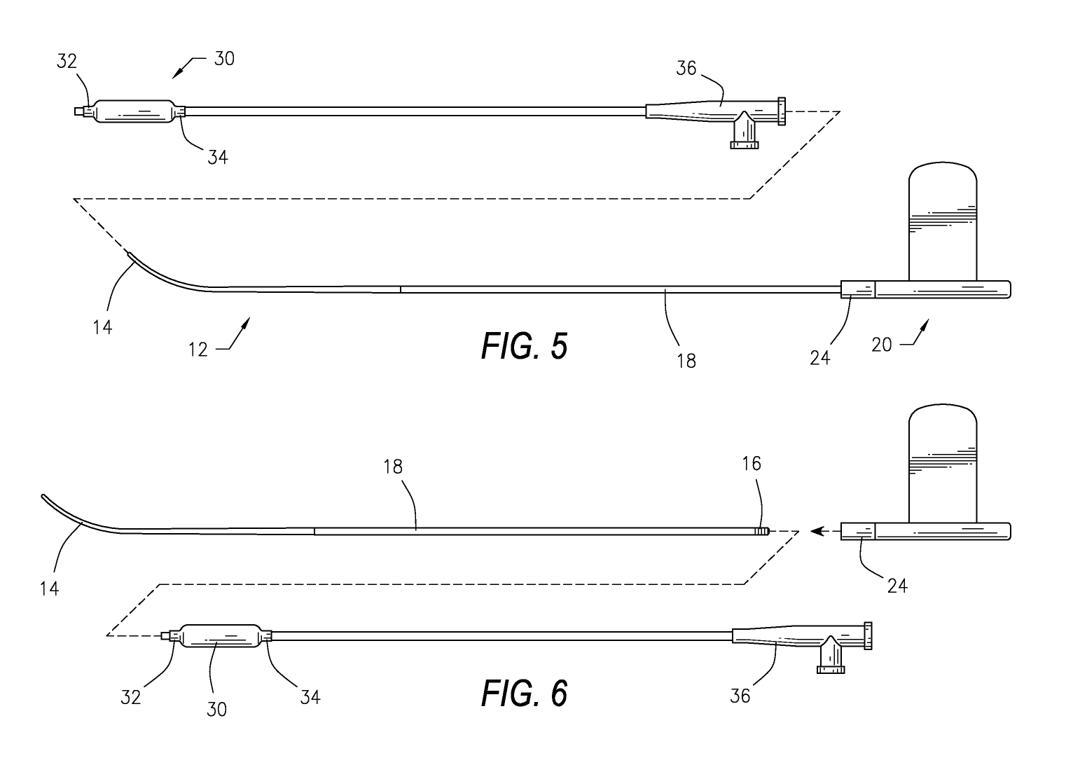

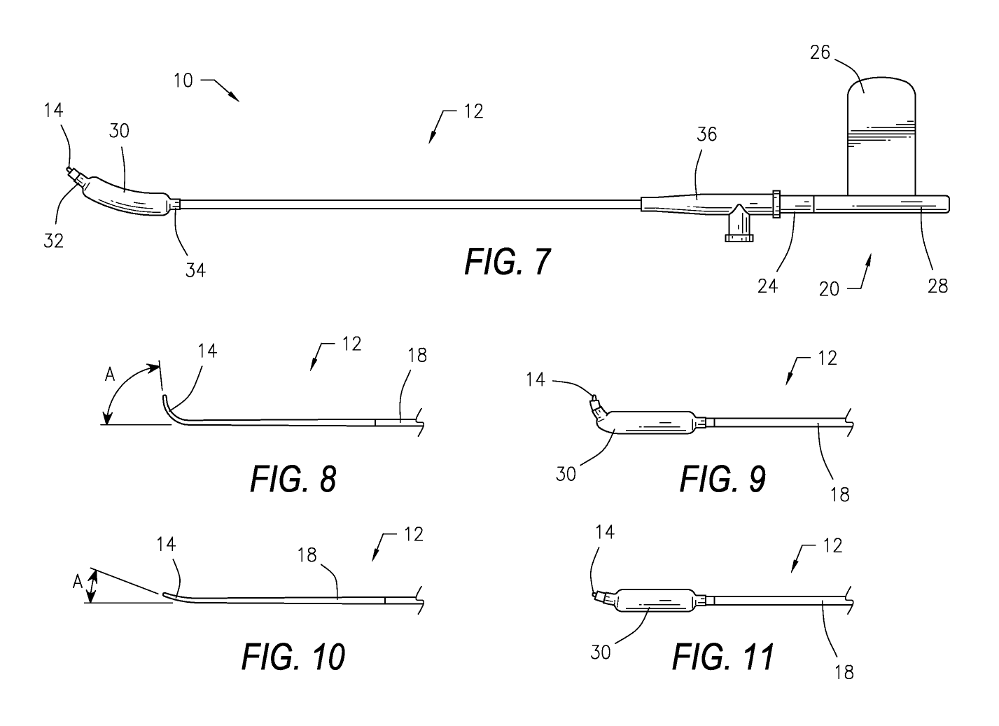

As can be seen in FIGS. 5 through 7, a disposable balloon 30 may be slipped directly over the guide seeker 12 and does not have to be removed or advanced, although the balloon 30 could be advanced if needed. The balloon 30 may be about 6 mm in diameter and about 18 mm long. The balloon 30 is positioned along the length L1 of the gentle curve of the bended terminal end 14, with a leading end 32 positioned about 1 mm from the tip 22 of the bending terminal end 14 and a terminal end 34 positioned along length L2 of the central grip 18 of the guide seeker 12. As illustrated in FIG. 5, the balloon 30 is slipped over the bended terminal end 14 of the guide seeker 12 while in FIG. 6 the balloon 30 is slipped over the attachment terminal end 16 of the guide seeker 12. Once the balloon 30 is seated over the guide seeker 12 as depicted in FIG. 7, a suitable air supply (not shown) may be fluidly coupled to an attachment mechanism 36 of the disposable balloon 30.

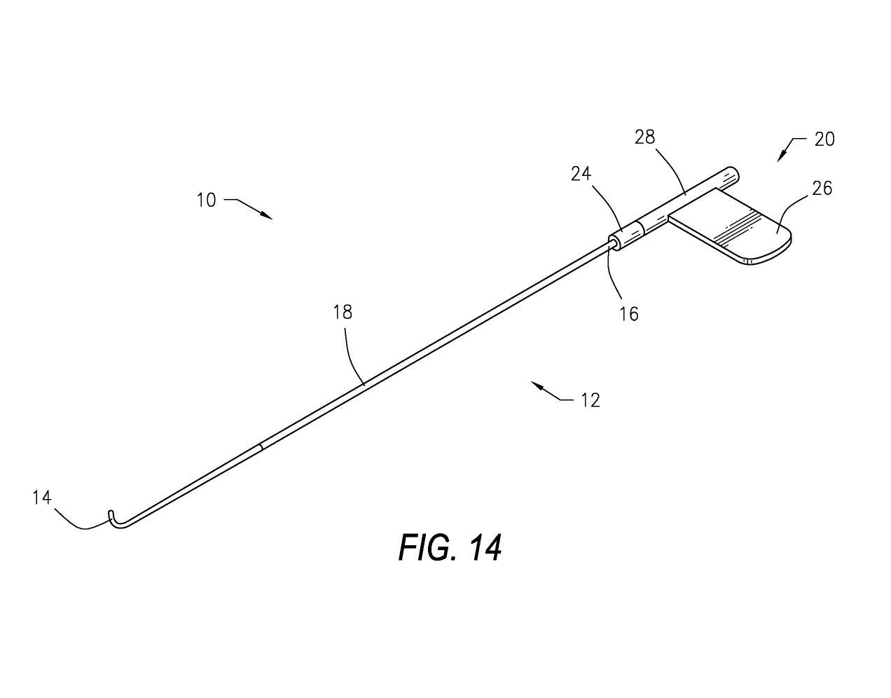

Referring now to FIGS. 8, 9 and 14, the reusable sinus dilation instrument 10 includes a maxillary sinus guide seeker 12 having the bended terminal end 14 with a rounded approximately 95 degree angle A. Similarly to the front sinus guide seeker discussed above, the bended terminal end 14 of the maxillary sinus seeker 12 may be approximately 8 mm in length L1 from the central grip 18. The diameter of the bended terminal end 14 is about 1 mm and progressively increases to about 2 mm in diameter for a length of about 8 cm along the central grip 18 of the guide seeker 12. The handle 20 is coupled to the attachment terminal end 16 of the guide seeker 12 and increases the diameter to about 5 mm and is about 2 cm in length. The wing 26 of the handle 30 may be about 2 mm in thickness, about 3.5 cm long, about 2 cm wide and about 23 cm from the tip 22 of the bended terminal end 14 of the guide seeker 12. As shown in FIG. 14, the handle 20 is disposed generally perpendicular to the bended terminal end 14 of the guide seeker 12. Although the handle 20 is exemplified as having a single wing 26 attached to and projecting from the body 28 of the handle 20, the handle 20 can include two (2) opposing wings 26 affixed to and protruding away from the body 28, and in such an embodiment, each of the wings 26 would be oriented generally perpendicular to the angle A of the bended terminal end 14 of the guide seeker 12.

Referring now to FIGS. 10 and 11, the reusable sinus dilation instrument 10 includes a sphenoid sinus guide seeker 12 having the bended terminal end 14 with a gentle curve having an approximately 10 degree angle A. The gentle curve may measure about 1 cm long and about 2 mm at the tip 22 of the bended terminal end 14 of the guide seeker 12. The diameter can be about 2 mm for a length of about 8 cm, and then increase to about 5 mm in diameter for about 2 cm in length. The wing 26 and the handle 20 of the sphenoid sinus guide seeker 20 may be configured and oriented similarly to the frontal sinus guild seeker 12, namely, the handle 20 is oriented generally coplanar with the bended terminal end 14 of the front sinus guide seeker 12. In addition, the handle 20 may be selectively positioned such that the wing 26 of the handle 20 is parallel to the bended terminal end 14 of the front sinus guide seeker 12 or such that the wing 26 of the handle 20 projects in a direction opposite of the curve of the bended terminal end 14 of the guide seeker 12.

Additionally, the handle 20 of the reusable sinus dilation instrument 10 may be specifically constructed for left or right sinus cavity procedures, and could be made with one wing 26 or with two (2) opposing wings 26. The balloon 30 may also have a locking mechanism (not shown) onto the handle 20, along with smaller wings 26 to allow advancement if needed.

The reusable sinus dilation instrument 10 may include a fiber optic element (not shown) embedded within a center channel (not shown) of the guide seeker 12 allowing the practitioner to confirm positioning and placement of the balloon 20 in the sinus cavity using transdermal illumination. With embedded fiber optics, the diameter of the tip 22 at the bended terminal end 14 of the guide seeker 12 increase to about 2 mm.

Whereas, the instruments and methods have been described in relation to the drawings, it should be understood that other and further modifications, apart from those shown or suggested herein, may be made within the scope of this invention.

What is claimed is:

1. A reusable sinus dilation instrument, comprising:
a guide seeker constructed from a sterilizable material, said guide seeker comprising a bended terminal end offset from an attachment terminal end, said guide seeker comprising a central grip intermediate of said terminal ends;
a removable handle attachable to said attachment terminal end at a predetermined orientation relative to said bended terminal end of said guide seeker.

2. The instrument of claim 1 wherein said handle is constructed of a sterilizable material.

3. The instrument of claim 2 wherein said sterilizable material of said guide seeker and/or said handle is stainless steel.

4. The instrument of claim 1 further comprising a disposable balloon capable of being slipped directly over said guide seeker.

5. The instrument of claim 1 wherein said removable handle is about 5 mm in diameter and about 2 cm in length.

6. The instrument of claim 1 wherein said handle is coupled to said attachment terminal end of said guide seeker using an attachment means selected from the group consisting of a threaded attachment or a luer taper fitting.

7. The instrument of claim 1 wherein said handle further comprises at least one wing attached to and protruding away from a body of said handle.

8. The instrument of claim 7 wherein said wing is about 2 mm thick, about 3.5 cm long and about 2 cm wide.

9. The instrument of claim 7 wherein said wing of said handle is oriented generally coplanar with and parallel to said bended terminal end of said front sinus guide seeker.

10. The instrument of claim 9 wherein said wing of said handle protrudes in a direction opposite of said bended terminal end of said guide seeker.

11. The instrument of claim 9 wherein said guide seeker is a frontal sinus guide seeker.

12. The instrument of claim 11 wherein said bended terminal end of said frontal sinus guide seeker has an angle of about 45 degrees with a long gentle curve of approximately 2.5 cm in length.

13. The instrument of claim 9 wherein said guide seeker is a sphenoid sinus guide seeker.

14. The instrument of claim 13 wherein said bended terminal end of said sphenoid guide seeker has a gentle curve with an approximately 10 degree angle.

15. The instrument of claim 7 wherein said wing of said handle is oriented generally perpendicular to said bended terminal end of said guide seeker.

16. The instrument of claim 15 wherein said guide seeker is a maxillary sinus guide seeker.

17. The instrument of claim 16 wherein said bended terminal end has a rounded approximately 95 degree angle.

18. The instrument of claim 16 wherein said handle further comprises two (2) opposing wings affixed to and protruding away from said handle, and each of said wings oriented generally perpendicular to said angle of said bended terminal end of said guide seeker.

19. The instrument of claim 7 wherein said at least one wing further comprises a pair of wings attached to and protruding away from opposing sides of said body of said handle.

20. The instrument of claim 1 wherein a tip of said bended terminal end is approximately 1 mm in diameter and progressively enlarges to about 2 mm in diameter over a length of about 8 cm.
